(19) United States
(12) Patent Application Publication
(10) Pub. No.: US 2019/0328317 A1
(43) Pub. Date: Oct. 31, 2019

(54) ULTRASOUND DIAGNOSTIC APPARATUS AND PUNCTURE ASSISTING METHOD

(71) Applicant: Canon Medical Systems Corporation, Otawara-shi (JP)

(72) Inventors: Fumio MOCHIZUKI, Utsunomiya (JP); Koichiro KURITA, Nasushiobara (JP); Takashi MASUDA, Utsunomiya (JP)

(73) Assignee: Canon Medical Systems Corporation, Otawara-shi (JP)

(21) Appl. No.: 16/392,102

(22) Filed: Apr. 23, 2019

(30) Foreign Application Priority Data
Apr. 27, 2018 (JP) 2018-086957

(51) Int. Cl. A61B 5/00; A61B 8/08; A61B 8/00

(52) U.S. Cl. CPC A61B 5/489; A61B 8/08; A61B 8/5246; A61B 8/488; A61B 8/463

(57) ABSTRACT

According to one embodiment, an ultrasound diagnostic apparatus includes an ultrasound probe and processing circuitry. The ultrasound probe is adapted to be applied on a surface of a subject. The ultrasound probe performs ultrasound scanning for a scan region in the subject. The processing circuitry analyzes, among a result of the ultrasound scanning, a part corresponding to a central portion of the scan region to calculate a distance between a blood vessel in the central portion and the surface. The processing circuitry causes a display to display the distance, a value based on the distance, or both the distance and the value.

### CROSS-REFERENCE TO RELATED APPLICATIONS

This application is based upon and claims the benefit of priority from prior Japanese Patent Application No. 2018-086957, filed Apr. 27, 2018, the entire contents of which are incorporated herein by reference.

### FIELD

Embodiments described herein relate generally to an ultrasound diagnostic apparatus and a puncture assisting method.

### BACKGROUND

An ultrasound diagnostic apparatus is adapted to generate ultrasound images by emitting ultrasound waves toward an object and receiving the reflected ultrasound waves using an ultrasound probe including multiple, orderly arranged ultrasound transducers.

Use of such an ultrasound diagnostic apparatus in puncturing a central vein is a recent trend that takes into account the safety during treatment operations. The central-vein puncture under the ultrasound guidance proceeds with obtaining the distance between a target blood vessel and the ultrasound probe by referring to, for example, a short-axis image of the blood vessel. Subsequently, a puncture needle is inserted into the subject at an angle of 45° with respect to the subject's skin and along the extension of the blood vessel, from the point on the subject's body surface which is away from the ultrasound probe by the same distance as the obtained distance, so that the puncture needle punctures the blood vessel as targeted.

This central-vein puncture under the ultrasound guidance, however, often incurs situations such as displacement between the center of an ultrasound probe and a target blood vessel. When such a situation occurs, referential images would not permit correctly locating the blood vessel, resulting in the inserted needle missing the blood vessel, or the images could misinform the depth of the blood vessel, resulting in a too deep puncture by the needle, and failure of such kinds in treatment procedures may be expected.

### BRIEF DESCRIPTION OF THE DRAWINGS

FIG. 1 is a block diagram showing a configuration of an ultrasound diagnostic apparatus according to a first embodiment.

FIG. 2 is a flowchart showing operations of the processing circuitry in FIG. 1 for displaying images for assisting a puncture.

FIG. 3 is a diagram pertaining to the processing for acquiring Doppler data for a blood vessel in a region of interest (ROI).

FIG. 4 is a diagram pertaining to the processing for calculating, based on the Doppler data acquired for the blood vessel in the ROI, a distance to the center of the blood vessel.

FIG. 5 is a diagram showing a tomographic image displayed on the display device in FIG. 1.

FIG. 6 is a diagram pertaining to the processing for calculating, based on the Doppler data acquired for the blood vessel in FIG. 5, a distance to the center of the blood vessel.

FIG. 7 is a diagram showing a B-mode short-axis image displayed on the display device in FIG. 1.

FIG. 8 is a diagram pertaining to the processing for calculating, based on the B-mode data acquired for the blood vessel in FIG. 7, a distance to the center of the blood vessel.

FIG. 9 is a diagram showing a tomographic image displayed on the display device in FIG. 1.

FIG. 10 is a diagram showing a display on the display device, not combined with a Doppler image.

FIG. 11 is a diagram showing a display on the display device, in which a needle's puncture length is presented together with a measurement value.

FIG. 12 is a diagram showing an example of how the needle's puncture length is calculated.

FIG. 13 is a diagram showing another example of calculating the needle's puncture length.

FIG. 14 is a diagram showing yet another example of calculating the needle's puncture length.

FIG. 15 is a diagram showing a display on the display device under a biplane mode.

FIG. 16 is a diagram showing a sectional image along a plane B before correction.

FIG. 17 is a diagram showing a sectional image along the plane B after the correction.

FIG. 18 is a diagram showing a display after removal of color representation of a Doppler image from an area surrounding the tip of a needle.

FIG. 19 is a block diagram showing a configuration of an ultrasound diagnostic apparatus according to a second embodiment.

FIG. 20 is a diagram showing an assist image generated by the assist image generating function in FIG. 19.

FIG. 21 is a diagram showing another example of the assist image generated by the assist image generating function in FIG. 19.

FIG. 22 is a diagram showing a display after removal of color representation of a Doppler image from a predetermined range surrounding the tip of a needle.

### DETAILED DESCRIPTION

In general, according to one embodiment, an ultrasound diagnostic apparatus includes an ultrasound probe and processing circuitry. The ultrasound probe is adapted to be applied on a surface of a subject. The ultrasound probe performs ultrasound scanning for a scan region in the subject. The processing circuitry analyzes, among a result of the ultrasound scanning, a part corresponding to a central portion of the scan region to calculate a distance between a blood vessel in the central portion and the surface. The processing circuitry causes a display to display the distance, a value based on the distance, or both the distance and the value.

Now, embodiments will be described with reference to the drawings.

### First Embodiment

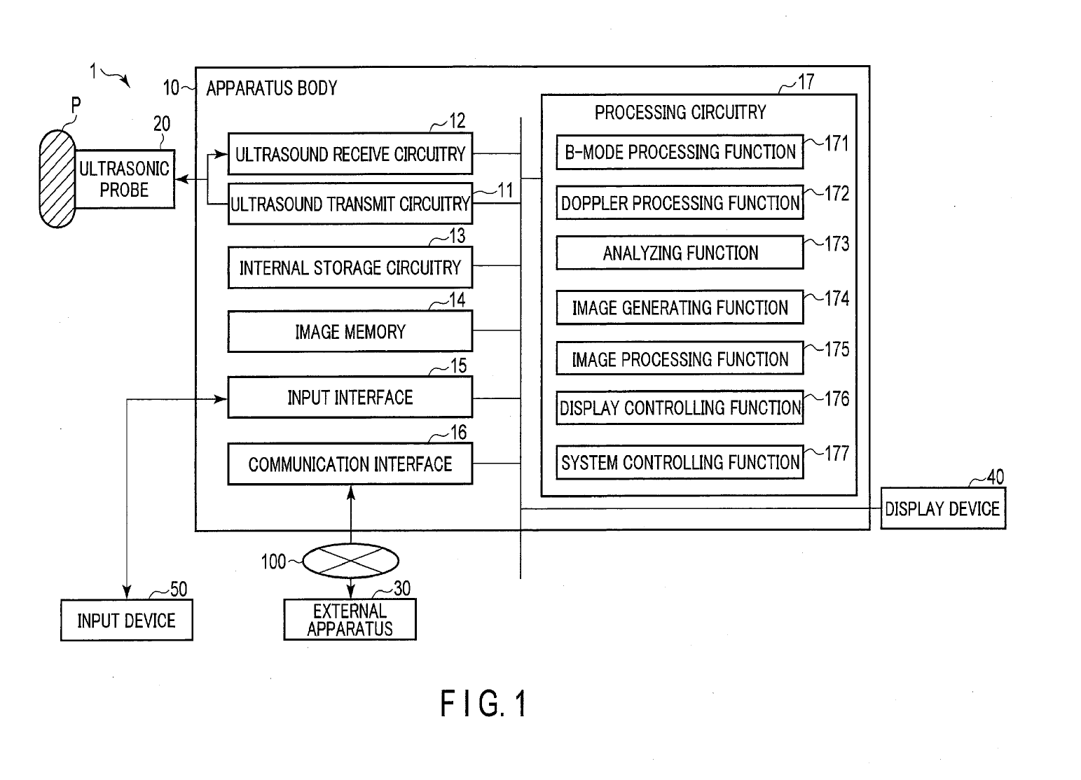

FIG. 1 is a block diagram showing an exemplary configuration of an ultrasound diagnostic apparatus 1 according to the first embodiment. As shown in FIG. 1, the ultrasound diagnostic apparatus 1 includes an apparatus body 10 and an ultrasound probe 20. The apparatus body 10 is connected to an external apparatus 30 via a network 100. The apparatus body 10 is also connected to a display device 40 and an input device 50.

The ultrasound probe 20 is adapted to perform ultrasound scanning for a scan region in a biological object P according to, for example, the control by the apparatus body 10. The ultrasound probe 20 includes, for example, multiple piezoelectric vibrators, a matching layer provided for the piezoelectric vibrators, and a backing member for preventing backward propagation of the ultrasound waves from the piezoelectric vibrators. Examples of the ultrasound probe 20 according to this embodiment include a one-dimensional array linear probe constituted by multiple ultrasound transducers arranged along a predetermined direction. The ultrasound probe 20 is adapted to be detachably connected to the apparatus body 10. The ultrasound probe 20 may include one or more buttons to be pressed for offset processing, freeze of an ultrasound image, etc.

The multiple piezoelectric vibrators generate ultrasound waves according to drive signals supplied from ultrasound transmit circuitry 11 in the apparatus body 10. The ultrasound probe 20 thus transmits the ultrasound waves to the biological object P. The ultrasound waves, upon transmission into the biological object P from the ultrasound probe 20, are reflected one after another by the discontinuous acoustic-impedance surfaces of the body tissues in the biological object P, and are received by the piezoelectric vibrators as reflected wave signals. The received reflected wave signals vary their amplitude depending on the difference in acoustic impedance between the discontinuous surfaces having reflected the ultrasound waves. When pulses of the transmitted ultrasound waves are reflected by a blood flow or a surface of the cardiac wall, etc. in motion, the resultant reflected wave signals involve a frequency shift attributable to the Doppler effect according to the moving object's velocity component in the direction of the ultrasound transmission. The ultrasound probe 20 converts the reflected wave signals received from the biological object P into electrical signals.

Note that what is shown in FIG. 1 is an example that assumes only the connection relationship between the ultrasound probe 20 for use in imaging and the apparatus body 10. However, the apparatus body 10 may be adapted for concurrent connections with multiple ultrasound probes. Which of the connected ultrasound probes will be used in imaging can be discretionarily selected by switchover operations.

The apparatus body 10 shown in FIG. 1 is an apparatus adapted to generate ultrasound images based on the reflected wave signals received by the ultrasound probe 20. The apparatus body 10, as shown in FIG. 1, includes the aforementioned ultrasound transmit circuitry 11, as well as ultrasound receive circuitry 12, internal storage circuitry 13, an image memory (cine-memory) 14, an input interface 15, a communication interface 16, and processing circuitry 17.

The ultrasound transmit circuitry 11 is a processor for supplying drive signals to the ultrasound probe 20. The ultrasound transmit circuitry 11 is realized by, for example, a trigger generating circuit, a delay circuit, a pulsar circuit, etc. The trigger generating circuit is adapted to repeat, at a predetermined rate frequency, generation of a rate pulse for forming an ultrasound wave for transmission. The delay circuit is adapted to apply a delay time to each rate pulse generated by the trigger generating circuit. The delay time is intended for respective piezoelectric vibrator and required for setting the transmission directivity by converging the ultrasound waves output from the ultrasound probe 20 into a beam shape. The pulsar circuit is adapted to apply the drive signals (drive pulses) to the piezoelectric vibrators in the ultrasound probe 20 at the timings based on the rate pulses. Controlling the delay circuit to vary the delay time applied to each rate pulse enables free adjustment of the direction of transmission from the face of the piezoelectric vibrators.

The ultrasound receive circuitry 12 is a processor for performing various types of processing on the reflected wave signals received by the ultrasound probe 20 to generate receive signals. The ultrasound receive circuitry 12 is realized by, for example, an amplifier circuit, an A/D converter, a receive delay circuit, an adder, etc. The amplifier circuit is adapted to perform gain correction by amplifying the reflected wave signals received by the ultrasound probe 20 for each channel. The A/D converter is adapted to convert the reflected wave signals after the gain correction into digital signals. The receive delay circuit is adapted to apply a delay time to the digital signals as required for setting the receive directivity. The adder is adapted to add up the delay time-involving digital signals. The addition processing by the adder generates receive signals that highlight a component reflected from the direction corresponding to the receive directivity.

The internal storage circuitry 13 may include, for example, a processor-readable storage medium such as a magnetic or optical storage medium or a semiconductor memory. The internal storage circuitry 13 stores a program for implementing ultrasound transmission and reception, a program for assisting punctures, and so on. The internal storage circuitry 13 also stores various data sets including diagnostic information (e.g., patient ID, doctor's remarks, etc.), diagnostic protocols, transmit conditions, receive conditions, signal processing conditions, image generating conditions, image processing conditions, body-mark generating programs, display conditions, and conversion tables describing preset ranges of color data for use in image formulation for respective sites for diagnosis. For example, one or more of these programs and various data sets may be prestored in the internal storage circuitry 13, or they may be stored and distributed in the form of non-transitory storage media and then read from the media for installation in the internal storage circuitry 13.

The internal storage circuitry 13 is adapted to further store two-dimensional B-mode image data, two-dimensional Doppler image data, etc., generated at the processing circuitry 17, in response to operational inputs for storage given via the input interface 15. The internal storage circuitry 13 may also be adapted to transfer the stored data to the external apparatus 30 via the communication interface 16.

Note that the internal storage circuitry 13 may also be a CD-ROM Drive, a DVD Drive, and a drive unit adapted to read and write various information sets from and to portable storage media such as a flash memory, and so on. It is possible for the internal storage circuitry 13 to write the stored data to portable storage media so that the data will be stored in the external apparatus 30 via the portable storage media.

The image memory 14 may include, for example, a processor-readable storage medium such as a magnetic or optical storage medium or a semiconductor memory. The image memory 14 is adapted to store image data corresponding to multiple frames immediately preceding an operational input for a freeze given via the input interface 15. The image data stored in the image memory 14 will be used in, for example, a continuous display (cine-display) operation.

It is not a requisite to realize the internal storage circuitry 13 and the image memory 14 by respective storage units independent of each other. The internal storage circuitry 13 and the image memory 14 may be realized by a single storage unit. Also, the internal storage circuitry 13 and the image memory 14 may each be realized by multiple storage units.

The input interface 15 is adapted to accept a variety of instructions from an operator via the input device 50. The input device 50 may be, for example, a mouse, a keyboard, panel switches, slider switches, a trackball, a rotary encoder, an operation panel, and a touch command screen (TCS). The input interface 15 is, for example, connected to the processing circuitry 17 via a bus so that it converts operational instructions input by the operator into electric signals and outputs them to the processing circuitry 17. Note that the present embodiment does not limit the input interface 15 to a member for making connections to physical operational components such as a mouse and a keyboard. Examples of the input interface 15 also include a circuit that is adapted to receive electric signals corresponding to the operational instructions input from external input devices separate from the ultrasound diagnostic apparatus 1, and to output the electric signals to the processing circuitry 17.

The communication interface 16 is connected to the external apparatus 30 via the network 100, etc., so that data communication is performed with the external apparatus 30. The external apparatus 30 may be, for example, a database for a picture archiving and communication system (PACS) for managing data related to various medical images, an electronic chart system for managing electronic charts attached with medical images, and so on. The communication with the external apparatus 30 may adopt any standards including, as one example, the standard for digital imaging and communication in medicine (DICOM).

The processing circuitry 17 is, for example, a processor functioning as a center of the ultrasound diagnostic apparatus 1. The processing circuitry 17 executes the programs stored in the internal storage circuitry 13 to realize functions corresponding to the respective programs. The processing circuitry 17 has, for example, a B-mode processing function 171, a Doppler processing function 172, an analyzing function 173, an image generating function 174, an image processing function 175, a display controlling function 176, and a system controlling function 177.

The B-mode processing function 171 is a function for generating B-mode data based on the receive signals from the ultrasound receive circuitry 12. More specifically, with this B-mode processing function 171, the processing circuitry 17 performs, for example, envelope detection, logarithmic amplification, etc., for the receive signals passed on from the ultrasound receive circuitry 12 to generate data (B-mode data) that expresses signal intensity by the degree of brightness. The generated B-mode data is stored in a RAW data memory (not shown) as B-mode raw data on two-dimensional ultrasound scan lines (rasters).

The Doppler processing function 172 is a function for generating data (Doppler data) as an extraction of Doppler effect-based motion information of a moving object that is present within the region of interest (ROI) set in a scan region, and this data is generated through the frequency analysis of the receive signals passed on from the ultrasound receive circuitry 12. More specifically, with the Doppler processing function 172, the processing circuitry 17 generates, as the motion information of a moving object, Doppler data by estimating an average velocity, an average distribution, an average power, etc., for each of multiple sample points. The moving object here is, for example, a blood flow, tissue portions such as the heart wall, a contrast medium, etc. This embodiment assumes that the processing circuitry 17 generates the Doppler data by estimating the average velocity, the average distribution, the average power, etc., of a blood flow for each of multiple sample points, as the motion information of the blood flow (blood flow information). The generated Doppler data is stored in the RAW data memory (not shown) as Doppler raw data on the two-dimensional ultrasound scan lines.

Using the Doppler processing function 172, the processing circuitry 17 can implement a colored Doppler technique called color flow mapping (CFM). In the CFM, ultrasound waves are transmitted and received multiple times for multiple scan lines. The processing circuitry 17 with the Doppler processing function 172 applies a moving target indicator (MTI) filter to the data strings corresponding to the same location so that signals (clutter signals) attributable to stationary tissue or slowly moving tissue are suppressed and signals attributable to a blood flow are extracted. The processing circuitry 17 then estimates the blood flow information including the velocity, distribution, power, etc., of the blood flow from the extracted blood flow signals.

The analyzing function 173 is a function for analyzing, among the result of ultrasound scanning, a part corresponding to the central portion of a scan region. More specifically, the processing circuitry 17 with the analyzing function 173 calculates the distance between a blood vessel and a body surface by, for example, analyzing the Doppler data for the central portion of the scan region. Note that the processing circuitry 17 may calculate the distance between the blood vessel and the body surface by instead or optionally analyzing the B-mode data for the central portion of the scan region. The processing circuitry 17 may also calculate the distance between the blood vessel and the body surface by analyzing the Doppler data and the B-mode data in combination.

The image generating function 174 is a function for generating data of images based on the data generated by the B-mode processing function 171 and the Doppler processing function 172. For example, the processing circuitry 17 with the image generating function 174 generates image data for display by a conversion process (scan conversion) of converting scan line signal sequences from the ultrasound scanning into scan line signal sequences in a video format as represented by televisions, etc. More specifically, the processing circuitry 17 subjects the B-mode raw data stored in the RAW data memory to raw-pixel conversion, for example, a coordinate conversion according to the scan configuration of the ultrasound probe 20, to generate pixel-based two-dimensional B-mode image data.

Also, the processing circuitry 17 subjects the Doppler raw data stored in the RAW data memory to the raw-pixel conversion to generate two-dimensional Doppler image data providing the blood flow information in the form of images. The two-dimensional Doppler image data may be velocity image data, distribution image data, power image data, or image data including any combination thereof.

It is also possible for the processing circuitry 17 to synthetically combine each of the generated two-dimensional B-mode image data and the generated two-dimensional Doppler image data with text information, scale marks, body marks, etc., based on various parameters.

The image processing function 175 is a function for performing predetermined image processing on the two-dimensional B-mode image data and the two-dimensional Doppler image data. More specifically, and for example, the processing circuitry 17 with the image processing function 175 performs image processing (smoothing) for regenerating an image having an average brightness value using multiple image frames in the two-dimensional B-mode image data or the two-dimensional Doppler image data generated by the image generating function 174, image processing (edge enhancement) of using a differential filter within images, and so on.

The display controlling function 176 is a function for controlling how the two-dimensional B-mode image data and the two-dimensional Doppler image data generated or processed by the image processing function 175 are displayed on the display device 40. More specifically, and for example, the processing circuitry 17 with the display controlling function 176 synthetically combines the two-dimensional B-mode image data with an indication of a ROI for Doppler data collection. The processing circuitry 17, referring to the operator's instructions input from the input device 50, synthetically combines the two-dimensional B-mode image data with the two-dimensional Doppler image data at a corresponding portion in the two-dimensional B-mode image data. At this time, the processing circuitry 17 may adjust the opacity of the two-dimensional Doppler image data for combining, according to the operator's instructions.

The processing circuitry 17 may further synthetically combine the two-dimensional B-mode image data having been combined with the two-dimensional Doppler image data, with a measurement line and a measurement value. The measurement line is a line extending from the surface of the ultrasound probe 20 to the center of the blood vessel and lying on the scan line arranged for the central portion of the scan region. The measurement value indicates the distance from the surface of the ultrasound probe 20 to the center of the blood vessel along the measurement line. Note that the processing circuitry 17 may synthetically add the measurement line and the measurement value to the two-dimensional B-mode image data.

Also, the processing circuitry 17 subjects the two-dimensional B-mode image data, or the two-dimensional B-mode image data combined with the two-dimensional Doppler image data, to various types of processing such as dynamic range processing, brightness (luminance) processing, contrast processing, y curve correction, and RGB conversion, to convert this image data into video signals. The processing circuitry 17 causes the display device 40 to present a display based on the video signals. The processing circuitry 17 may optionally generate a user interface (graphical user interface: GUI) for prompting the operator to input various instructions from the input device 50, and cause the display device 40 to display the GUI. Examples which are appropriately adoptable as the display device 40 include a CRT display, a liquid crystal display, an organic EL display, an LED display, a plasma display, or any other display known in this technical field.

The system controlling function 177 is a function that takes total control over the processing performed by the ultrasound diagnostic apparatus 1. More specifically, the processing circuitry 17 with the system controlling function 177 controls the ultrasound transmit circuitry 11 and the ultrasound receive circuitry 12, as well as the functions of the processing circuitry 17, based on various setting commands input from the operator via the input device 50, and the various programs and data sets read from the internal storage circuitry 13.

For example, the processing circuitry 17 controls the ultrasound transmit circuitry 11 and the ultrasound receive circuitry 12 to cause the ultrasound probe 20 to perform ultrasound scanning. More specifically, and for example, the processing circuitry 17 sets a ROI for Doppler data collection based on the operator's instruction, in order to carry out the CFM. The processing circuitry 17 controls the ultrasound transmit circuitry 11 and the ultrasound receive circuitry 12 to cause the ultrasound probe 20 to perform ultrasound scanning for collecting Doppler data for the ROI. Also, the processing circuitry 17 controls the ultrasound transmit circuitry 11 and the ultrasound receive circuitry 12 to cause the ultrasound probe 20 to perform ultrasound scanning for collecting B-mode data for the region other than the ROI.

Next, description will be given of how the ultrasound diagnostic apparatus 1 configured as above operates for performing the central-vein puncture.

First, a physician as an operator places a patient in a body position suitable for the puncture. After the placement of the subject, the physician initiates a pre-scan operation for the vein using the ultrasound probe 20. The pre-scan operation includes scanning for collecting the B-mode data and the Doppler data. The B-mode data is collected for the scan region, and the Doppler data is collected for the ROI set within the scan region. The ultrasound waves transmitted into the patient from the ultrasound probe 20 are reflected by the discontinuous acoustic-impedance surfaces of the body tissues in the biological object P one after another, and received by the ultrasound probe 20 as reflected wave signals. The ultrasound receive circuitry 12 performs various types of processing on the reflected wave signals received by the ultrasound probe 20 to generate receive signals.

The processing circuitry 17 of the ultrasound diagnostic apparatus 1 generates, by its B-mode processing function 171, B-mode raw data on two-dimensional ultrasound scan lines, based on the receive signals from the ultrasound receive circuitry 12. The processing circuitry 17 with the image generating function 174 generates two-dimensional B-mode image data by subjecting the B-mode raw data to the raw-pixel conversion.

Also, the processing circuitry 17 with the. Doppler processing function 172 generates Doppler raw data on the ultrasound scan lines in the ROI, based on the receive signals from the ultrasound receive circuitry 12. The processing circuitry 17 with the image generating function 174 generates two-dimensional Doppler image data by subjecting the Doppler raw data to the raw-pixel conversion. The processing circuitry 17 with the display controlling function 175 synthetically combines the generated two-dimensional B-mode image data with the two-dimensional Doppler image data, and causes the display device 40 to display the synthesized image data as a tomographic image.

The physician checks arteries and veins using the tomographic image displayed according to the pre-scan operation, and estimates whether or not the intended vein is suited for a puncture. By way of example, the description will assume an instance where the physician selects the patient's internal jugular vein as a puncture target site. Note that the puncture target site in the central-vein puncture is not limited to an internal jugular vein, but any site may be selected as the puncture target site, such as a subclavian vein, a femoral vein, or a brachial basilic vein. The physician moves the ultrasound probe 20 while monitoring the tomographic image displayed on the display device 40 for the scan region, so that the tomographic image will turn the short-axis image of the internal jugular vein and also include the internal jugular vein at its central portion.

Upon selecting the internal jugular vein as the puncture target site, the physician, for example, instructs the ultrasound diagnostic apparatus 1 to run a puncture assisting program.

According to this instruction, the processing circuitry 17 of the ultrasound diagnostic apparatus 1 reads the puncture assisting program stored in the internal storage circuitry 13, and executes the read program. The execution of the puncture assisting program may be started at the time of the pre-scan operation.

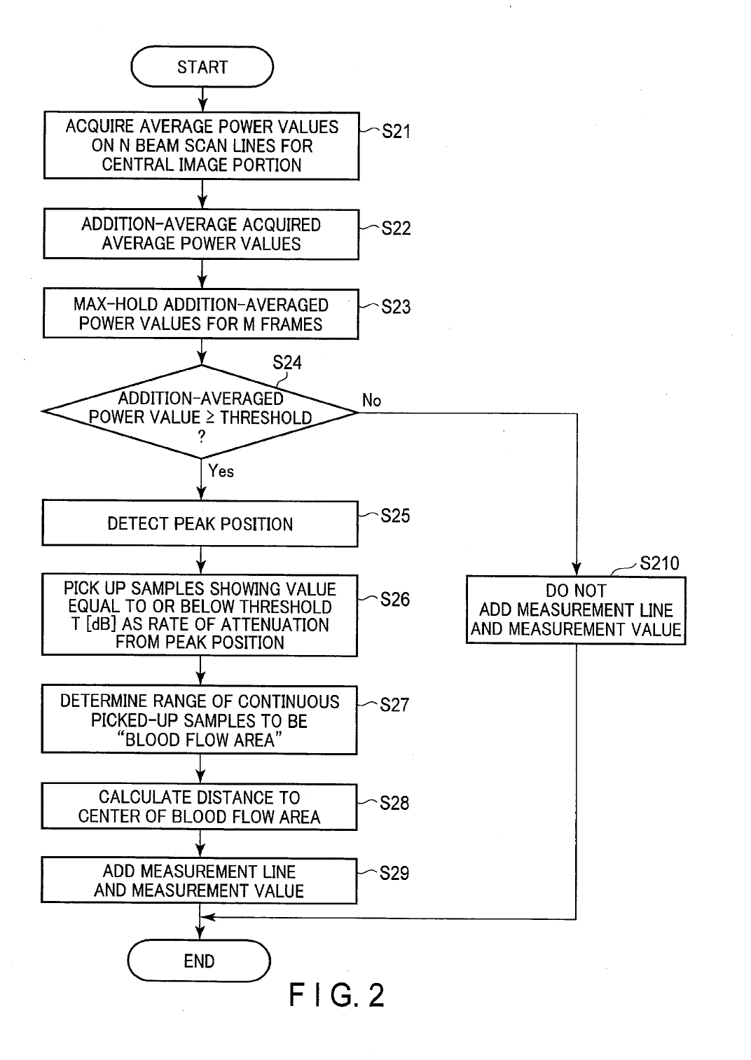

FIG. 2 is a flowchart showing exemplary operations of the processing circuitry 17 shown in FIG. 1, for displaying images for assisting a puncture. The processes shown in FIG. 2 are performed in a predetermined cycle, for example, at a frame period.

With the execution of the puncture assisting program, the processing circuitry 17 performs, for example, the analyzing function 173. The processing circuitry 17 with the analyzing function 173 acquires a set of average power values at respective sample points on each of N ultrasound scan lines positioned for the central portion of the scan region (step S21). The processing circuitry 17 adds up and averages values in the acquired average power value sets on the N ultrasound scan lines, for each sample point (step S22). The processing circuitry 17 retains sets of such addition-averaged power values for M frames, and outputs the maximum value for each sample point among the retained addition-averaged power value sets for M frames (step S23). The processing circuitry 17, upon calculating a new addition-averaged power value set, discards the oldest addition-averaged power value set while retaining this new value set.

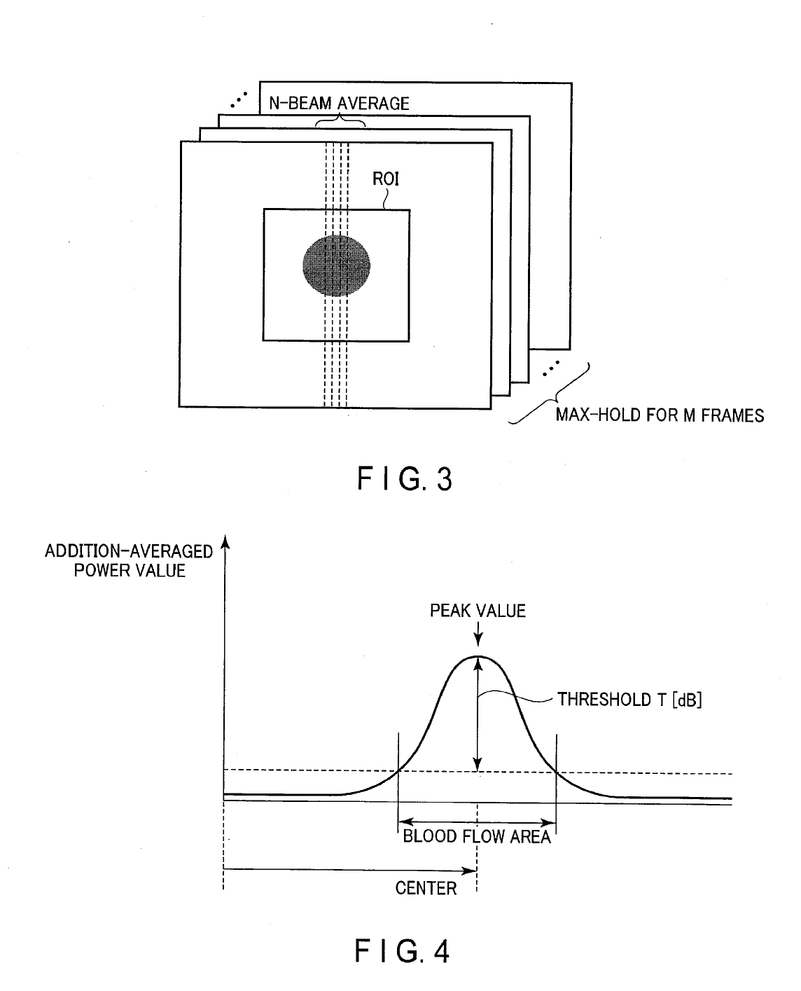

FIG. 3 is an exemplary schematic diagram pertaining to the processes of steps S21 to S23 for the internal jugular vein in the ROI. As in the example shown in FIG. 3, an average power value set is acquired on the scan line passing through the center of the internal jugular vein positioned in the central portion of the scan region. The acquired average power value set is then subjected to the addition-average calculations together with, for example, average power value sets on two scan lines on each of both sides of the center scan line. Accordingly, the maximum addition-averaged power values for the respective sample points, among the value sets obtained by the addition-average calculations for M frames, are output.

The processing circuitry 17 determines whether or not the output addition-averaged power values are equal to or greater than a preset threshold (step S24). If it is determined that the output addition-averaged power values are equal to or greater than the threshold (Yes in step S24), the processing circuitry 17 detects a peak value of the output addition-averaged power values and acquires a position in the depth direction, i.e., a peak position, where the detected peak value is measured (step S25).

The processing circuitry 17 extracts samples showing, as a rate of attenuation from the detected peak value, a value equal to or below a preset value T [dB], from the output addition-averaged power values (step S26). Note that the criteria for extracting samples are not limited to the attenuation rate. Samples showing a preset attenuation width value or below may be extracted. The processing circuitry 17 determines the range, in which the extracted samples are continuous, to be a “blood flow area” (step S27). The processing circuitry 17 calculates the distance (depth) from the surface of the ultrasound probe 20, i.e., the body surface, to the center of the “blood flow area” (step S28).

FIG. 4 is an exemplary schematic diagram pertaining to the processes of steps S25 to S28 for addition-averaged power values acquired for the internal jugular vein in the ROI. As in the example shown in FIG. 4, the peak value is detected from the output addition-averaged power values. Samples showing an attenuation rate of T [dB] or below from the detected peak value are extracted from the output addition-averaged power values and determined to be the “blood flow area”. The distance to the center of the “blood flow area” is then calculated.

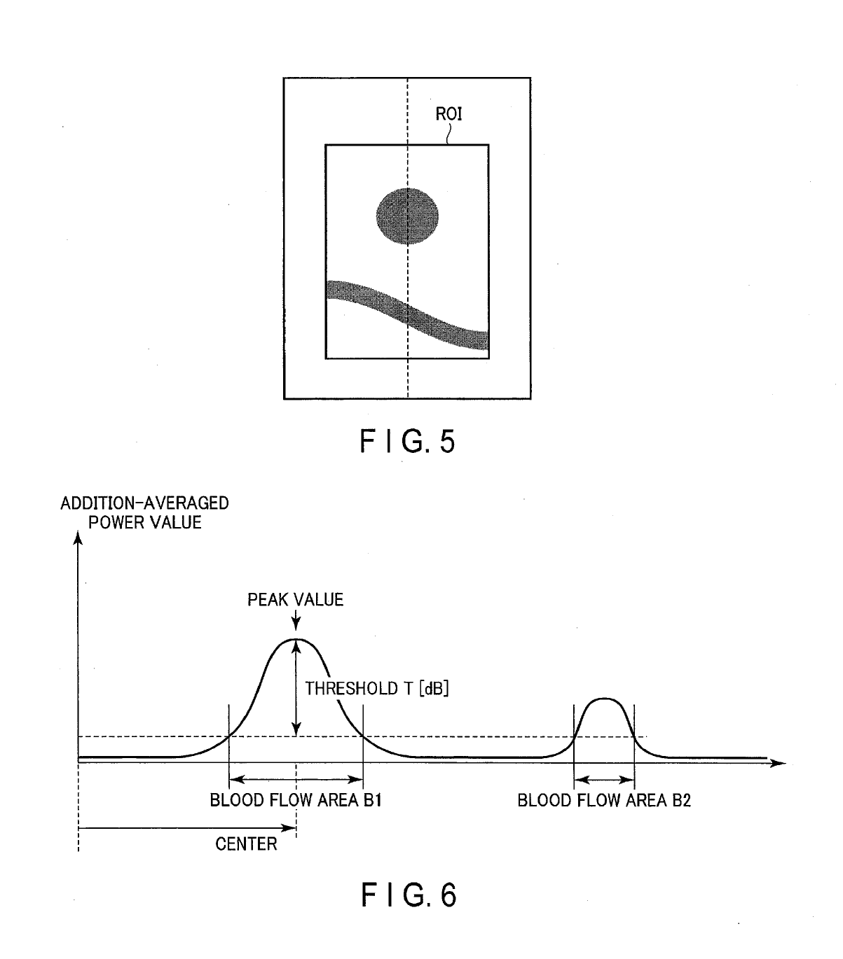

Depending on a puncture target site, the scan lines for the central portion of the scan region could cover more than one blood vessel, as shown in FIG. 5. In such an instance of the blood vessel presence as shown in FIG. 5, outputs given by the process of step S23 would be the addition-averaged power values as shown in, for example, FIG. 6. With the addition-averaged power values output as shown in FIG. 6, a blood flow area B1 and a blood flow area B2 are extracted by the processes of steps S25 to S27. Upon extraction of more than one blood flow area, the processing circuitry 17 adopts the one closer to the surface of the ultrasound probe 20, as a measurement target. In this case, thus, the blood flow area B1 is determined to be the measurement target, and the distance to its center is calculated.

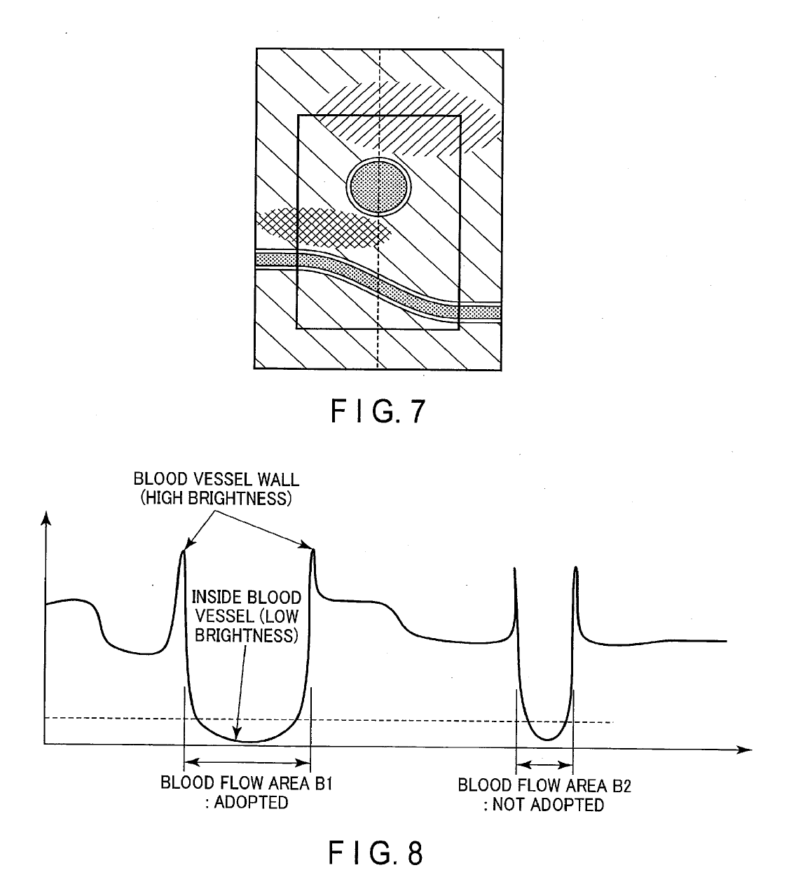

Note that it is not a limitation for the processing circuitry 17 with the analyzing function 173 to utilize Doppler data for performing the extraction of a blood flow area. For example, the analyzing function 173 may be a function for instead or optionally extracting a blood flow area by utilizing B-mode data. An instance will be assumed, where a B-mode image shown in FIG. 7 is displayed on the display device 40. The processing circuitry 17 in this instance acquires a set of brightness values on each of N ultrasound scan lines positioned for the central portion of the scan region. The processing circuitry 17 adds up and averages values in the acquired brightness value sets on the N ultrasound scan lines, for each sample point.

A wall of a blood vessel shows a brightness higher than other sites, and an inside of a blood vessel shows a brightness lower than other sites. The processing circuitry 17 extracts a blood flow area through detection of patterns including a high-to-low brightness transition pattern and a low-to-high brightness transition pattern, from the addition-averaged brightness values.

FIG. 8 is a schematic diagram showing an example of brightness values output based on the B-mode short-axis image shown in FIG. 7. As shown in FIG. 8, the wall portions of the blood vessels show brightness values higher than the brightness values of the other sites, and the inner portions of the blood vessels show brightness values lower than the brightness values of the other sites. From the output brightness values, the processing circuitry 17 detects a pattern in which the brightness transitions from a high value to a low value, and a pattern in which the brightness transitions from a low value to a high value. The detection allows for the extraction of a blood flow area B1 and a blood flow area B2 from the output brightness values. The processing circuitry 17 calculates the distance from the surface of the ultrasound probe 20, i.e., the body surface, to the center of the blood flow area B1 which is closer to the body surface.

The extraction of a blood flow area with the analyzing function 173 may be based on the combination of the analysis using Doppler data and the analysis using B-mode data. For example, the processing circuitry 17, in response to the coincidence between the blood flow area acquired based on Doppler data and the blood flow area acquired based on B-mode data, adopts the center of this blood flow area of coincidence.

Upon calculating the distance to the center of the “blood flow area”, the processing circuitry 17 performs the display controlling function 176. The processing circuitry 17 with the display controlling function 176 synthetically adds a measurement line and a measurement value to a tomographic image, i.e., an image constituted by the two-dimensional B-mode image data synthetically combined with the two-dimensional Doppler image data (step S29). The measurement line extends from the surface of the ultrasound probe 20 to the center calculated in step S28, and lies on the scan line arranged for the central portion of the scan region. The measurement value is indicative of the distance from the surface of the ultrasound probe 20 to the center calculated in step S28.

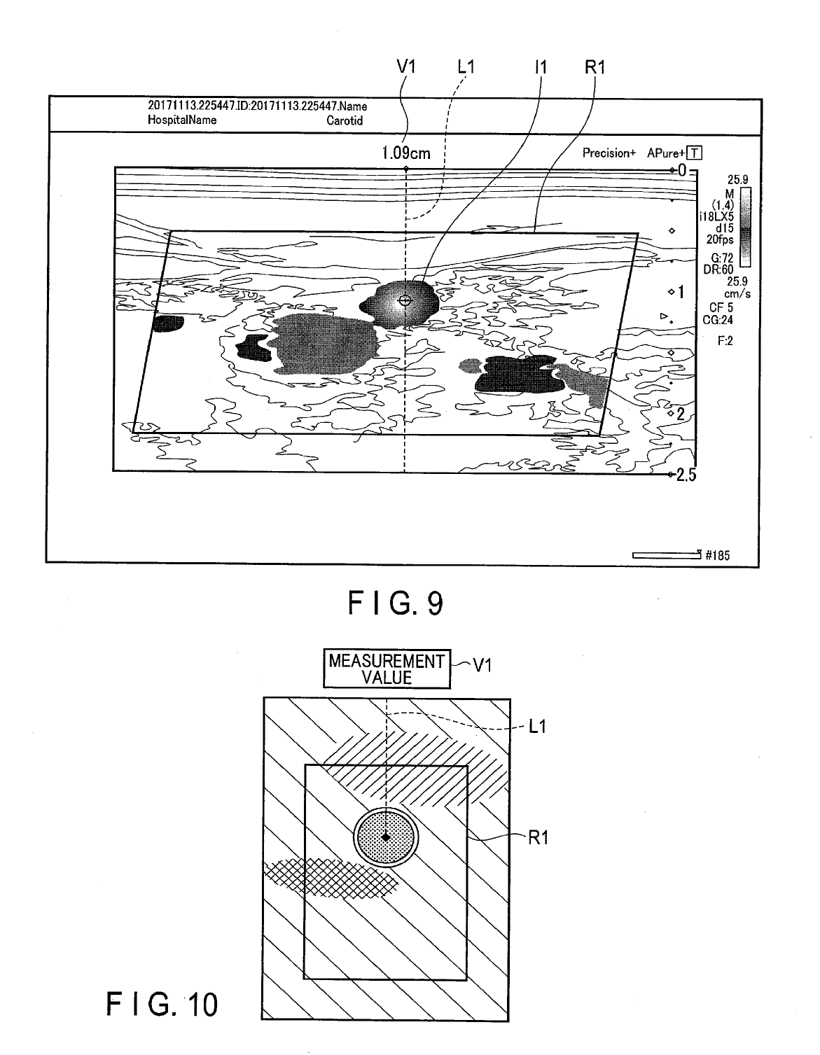

FIG. 9 is a diagram showing an example of the tomographic image displayed on the display device 40 in FIG. 1. As shown in FIG. 9, a Doppler image I1 for the internal jugular vein is displayed within a ROI indication R1. Also, a measurement line L1 is displayed as a line extending from the center of the Doppler image I1 to the surface of the ultrasound probe 20, and a measurement value V1 is displayed right above the point at which the surface of the ultrasound probe 20 intersects the measurement line L1.

The example shown in FIG. 9 is a display in which the Doppler image I1 has a high opacity. Note that if it is desired to view the tip of a puncture needle in a B-mode image, the Doppler image combination may be omitted, or the opacity of the Doppler image may be lowered. FIG. 10 is a schematic diagram showing an example of a display on the display device 40, without the Doppler image combination. In this case, as shown in FIG. 10, the measurement line L1 is displayed as a line extending from the center of the B-mode short-axis image presented within the ROI indication R1 to the surface of the ultrasound probe 20, and the measurement value V1 is displayed right above the point at which the surface of the ultrasound probe 20 intersects the measurement line L1.

If it is determined in step S24 that the output addition-averaged power values fall below the threshold (No in step S24), the processing circuitry 17 forgoes addition of the measurement line and the measurement value (step S210) and terminates the processing.

Upon confirming the measurement line and the measurement value displayed on the tomographic image, the physician inserts a puncture needle into the subject according to the display. At this time, the physician inserts the puncture needle at an angle of 45° with respect to the subject's skin and along the extension of the blood vessel, from the point on the subject's body surface which is away from the ultrasound probe 20 by the same distance as the distance verified by the measurement value. The physician is therefore enabled to puncture the targeted blood vessel. Additionally, if insertion angles other than 45° are desired, for example, 60°, 30°, and so on, the insertion point for the puncture needle is adjusted to be away from the ultrasound probe 20 by the distance according to the selected insertion angle.

According to the first embodiment as in the foregoing description, the ultrasound probe 20 carries out ultrasound scanning for the scan region in the body of a subject. The processing circuitry 17 of the ultrasound diagnostic apparatus 1 analyzes the result of the ultrasound scanning for the part corresponding to the central portion of the scan region, so that it calculates the distance between the blood vessel within the central portion and the body surface. The processing circuitry 17 then causes the display device 40 to display the calculated distance in real time. The ultrasound diagnostic apparatus 1 can therefore keep a physician from making an error in specifying the depth of a puncture.

According to the first embodiment, also, the processing circuitry 17 calculates the distance between the blood vessel and the body surface by utilizing the Doppler data on the scan lines arranged for the central portion of the scan region. The ultrasound diagnostic apparatus 1 can therefore perform calculation for providing an accurate distance between the blood vessel and the body surface.

According to the first embodiment, furthermore, the processing circuitry 17 causes the display device 40 to display the measurement line that connects the ultrasound probe 20 and the blood vessel along the line passing through both of their centers. The ultrasound diagnostic apparatus 1 can therefore prevent a puncture operation from being performed while the center of the ultrasound probe 20 and the blood vessel are displaced from each other.

Yet more, according to the first embodiment, the processing circuitry 17 does not cause the display device 40 to display the measurement line and the measurement value if the acquired average power values are smaller than the preset value. This forgoes the display of the measurement line and the measurement value on the display device 40 when the central portion of the scan region does not cover the blood vessel. The ultrasound diagnostic apparatus 1 can therefore inform a physician of a state of displacement between the center of the ultrasound probe 20 and the blood vessel.

Note that the ultrasound diagnostic apparatus 1 according to the first embodiment is not limited to the foregoing description. For example, the description of the embodiment has assumed the instances where the processing circuitry 17 uses both or at least either of the B-mode data and the Doppler data before scan conversion, in order to calculate the distance between the blood vessel and the body surface. However, this does not pose a limitation. The processing circuitry 17 may also calculate the distance between the blood vessel and the body surface, by instead using both or at least either of the B-mode data and the Doppler data having been scan-converted into the scan line signal sequences in a video format.

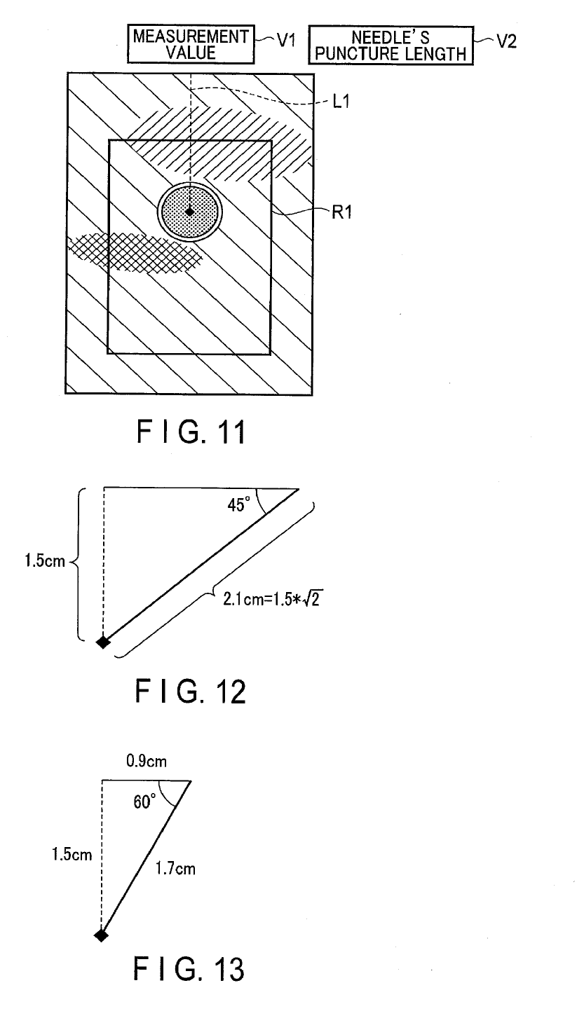

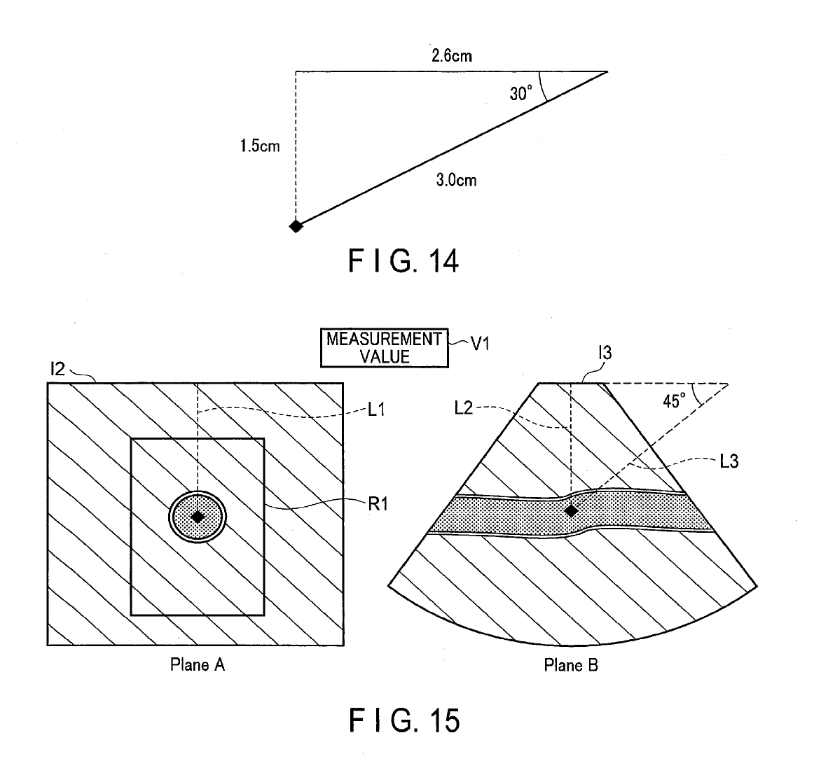

Also, the description of the above embodiment has assumed the instances where the measurement value is synthetically added to the tomographic image or the B-mode image. However, the embodiment is not limited to these instances. For example, the processing circuitry 17 may synthetically add a value that can be calculated based on the distance between the blood vessel and the body surface, in place of or in addition to the measurement value. Examples of the value that can be calculated based on the distance between the blood vessel and the body surface include the length required of a puncture needle for the puncture operation. FIG. 11 is a schematic diagram showing an example of a display on the display device 40, in which the needle's puncture length is presented together with the measurement value. As shown in FIG. 11, a needle's puncture length V2 is displayed next to the measurement value V1. For example, when the puncture needle is inserted at an angle of 45° with respect to the skin, the needle's puncture length is calculated to be a value of the distance between the blood vessel and the body surface, multiplied by √2, as shown in FIG. 12. Also, when the puncture needle is inserted at an angle of 60° with respect to the skin, the needle's puncture length is calculated to be a value of the distance multiplied by 2/√3, as shown in FIG. 13. When the puncture needle is inserted at an angle of 30° with respect to the skin, the needle's puncture length is calculated to be a value of the distance multiplied by 2, as shown in FIG. 14.

The description of the above embodiment has assumed the instances where the ultrasound probe 20 is provided with one or more buttons to be pressed for offset processing, freeze of an ultrasound image, etc. The button components which can be furnished on the ultrasound probe 20 are not limited to these. For example, the ultrasound probe 20 may also include one or more selector switches for controlling whether or not to combine the tomographic image or the B-mode image with the measurement value and the measurement line. For example, the physician may press down such a selector switch button when the presentation of the measurement value or the measurement line is no longer desired, so that the measurement value and the measurement line displayed on the tomographic image or the B-mode image can disappear.

Also, the ultrasound probe 20 may include one or more hold buttons for maintaining the presentation of the measurement value and the measurement line on the tomographic image or the B-mode image as currently displayed. For example, the physician may press down such a hold button under the situation of unstable detection for the blood vessel center due to large pulsating characteristics of the blood flow, so that the presentation of the measurement value and the measurement line as of the press-down of the button can be maintained on the display screen. The ultrasound probe 20 may of course include a release button together with the hold button, for canceling the hold of the display on the display screen.

### Other Implementation Examples

The description of the first embodiment has assumed the instances where the ultrasound probe 20 is a one-dimensional array linear probe. However, this is not a limitation. The ultrasound probe 20 may be a probe of any configuration, in particular, a two-dimensional array linear probe constituted by multiple ultrasound transducers arranged in two-dimensional matrix. In this case, the processing circuitry 17 with the B-mode processing function 171 generates B-mode raw data on three-dimensional ultrasound scan lines, based on the receive signals of three-dimensional features from the ultrasound receive circuitry 12. Also, the processing circuitry 17 with the Doppler processing function 172 generates Doppler raw data on the three-dimensional ultrasound scan lines, based on the receive signals of three-dimensional features from the ultrasound receive circuitry 12.

The processing circuitry 17 with the analyzing function 173 analyzes, for example, a part of the result of the ultrasound scanning, which corresponds to the central portion of the three-dimensional scan region. More specifically, the processing circuitry 17 calculates the distance between the blood vessel and the body surface by, for example, analyzing the Doppler data on the scan lines for the central portion of the three-dimensional scan region. The processing circuitry 17 may calculate the distance between the blood vessel and the body surface by instead or optionally analyzing the B-mode data for the central portion of the three-dimensional scan region. Also, the processing circuitry 17 may perform the analyzing function 173 after performing the scan conversion with the image generating function 174.

The processing circuitry 17 with the image generating function 174 generates three-dimensional B-mode image data constituted by voxels in an intended range, by subjecting the three-dimensional B-mode raw data to raw-voxel conversion. Also, the processing circuitry 17 with the image generating function 174 generates three-dimensional Doppler image data constituted by voxels in an intended range, by subjecting the three-dimensional Doppler raw data to the raw-voxel conversion.

The processing circuitry 17 further realizes image processing functions by executing the programs stored in the internal storage circuitry 13. The processing circuitry 17 with the image processing functions performs rendering processing so that the display device 40 can provide two-dimensional displays of the three-dimensional B-mode image data and the three-dimensional Doppler image data. Examples of the rendering processing include volume rendering, surface rendering, multi-planner reconstruction (MPR), and so on.

For example, upon receipt of an instruction for setting a biplane mode via the input device 50, the processing circuitry 17 generates a first sectional image for a plane A and a second sectional image for a plane B orthogonal to the plane A, based on the three-dimensional B-mode image data and the three-dimensional Doppler image data. In this implementation example of the embodiment, the plane A represents a plane formed along the direction in which the ultrasound transducers of the ultrasound probe 20 are arranged, so the first sectional image shows the short-axis image of the blood vessel. The plane B represents a plane vertical to the arrangement direction of the ultrasound transducers of the ultrasound probe 20, so the second sectional image shows the long-axis image of the blood vessel.

The processing circuitry 17 then uses the display controlling function 176 to, for example, display the first sectional image and the second sectional image in parallel positions, and to synthetically combine the first sectional image with an indication of the ROI and a first measurement line extending from the surface of the ultrasound probe 20 to the center of the blood vessel. Furthermore, the processing circuitry 17 may synthetically combine the second sectional image with, for example, a second measurement line corresponding to the first measurement line, and a guide line crossing this second measurement line and indicative of an insertion route for the puncture needle. The guide line is obtained from, for example, the relationship between the second measurement line and the insertion angle of the puncture needle. The processing circuitry 17 may also display the measurement value, for example, between the first sectional image and the second sectional image, for indicating the distance from the surface of the ultrasound probe 20 to the center of the blood vessel.

FIG. 15 is a schematic diagram showing an example of a display on the display device 40 under a biplane mode. As in FIG. 15, a first sectional image I2 along the plane A shows a first measurement line L1, i.e., a line extending from the center of the B-mode short-axis image given within the ROI indication R1 to the surface of the ultrasound probe 20. Also, a second sectional image R3 along the plane B shows a second measurement line L2 corresponding to the first measurement line L1, and a guide line L3 joining the second measurement line L2 and indicating the insertion route for the puncture needle. Also, there is a display of a measurement value V1 between the first sectional image I2 and the second sectional image I3.

Note that what is shown in FIG. 15 is an example where neither the first sectional image I2 nor the second sectional image I3 is combined with a Doppler image. This is not a limitation. The processing circuitry 17 with the display controlling function 176 may synthetically combine the B-mode image with a Doppler image in one or both of the first sectional image I2 and the second sectional image I3.

The plane B displayed under the biplane mode is not limited to the plane vertical to the arrangement direction of the ultrasound transducers of the ultrasound probe 20. As the plane B, the processing circuitry 17 may also adopt a plane along the direction in which the puncture needle advances within the body of the subject.

More specifically, the processing circuitry 17 with the image processing functions generates sectional images respectively for a plane orthogonal to the plane A and multiple planes each inclined at a predetermined angle from the orthogonal plane.

The processing circuitry 17 with the analyzing function 173 then sets, for each of the generated multiple sectional images, the second measurement line corresponding to the first measurement line synthetically added to the first sectional image along the plane A. The processing circuitry 17 sets the guide line crossing the second measurement line and indicative of the insertion route for the puncture needle, for each of the multiple sectional images. The processing circuitry 17 calculates the sum of the brightness values on the set guide line, for each of the multiple sectional images. The processing circuitry 17 adopts the plane that gives the sectional image having the largest brightness sum value, as the plane B.

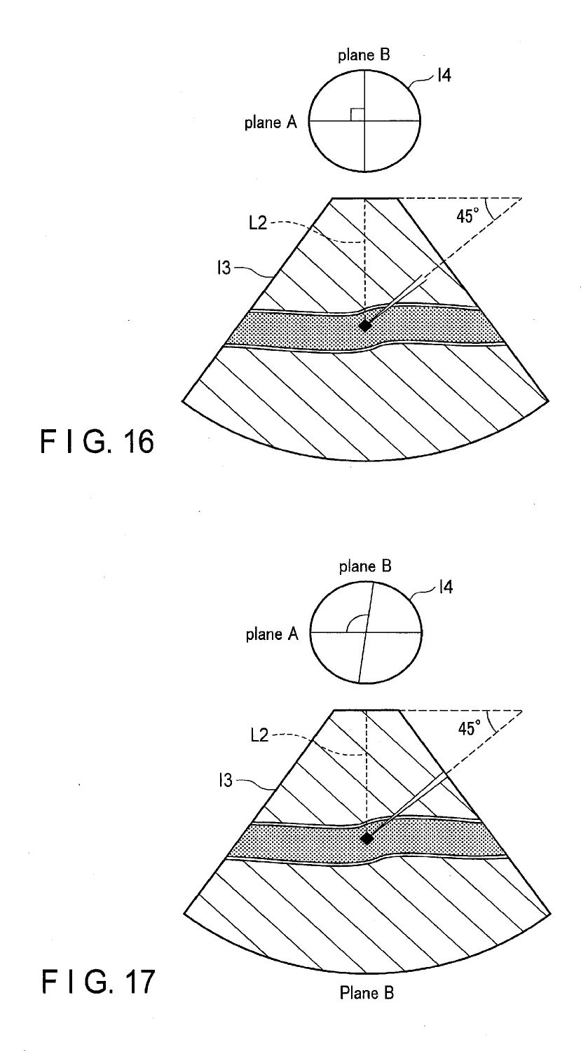

FIGS. 16 and 17 are schematic diagrams pertaining to an instance where the set angle of the plane B is corrected. FIGS. 16 and 17 each show the second sectional image I3 along the plane B, and an angle icon image I4. The second sectional image I3 and the angle icon image I4 shown in FIG. 16 are in the state before the correction to the plane B's set angle, that is, the state where the plane B is orthogonal to the plane A. The second sectional image I3 and the angle icon image I4 shown in FIG. 17 are in the state after the correction to the plane B's set angle, that is, the state where the plane B is inclined at an angle of 90°+X° from the plane A.

In this instance, for example, the processing circuitry 17 generates sectional images respectively for a plane orthogonal to the plane A and multiple planes each inclined at ±X° from the orthogonal plane. The processing circuitry 17 sets the guide line L3 and calculates the sum of the brightness values on the set guide line L3, for each of the generated multiple sectional images. The processing circuitry 17 adopts the angle X° that has provided the sectional image having the largest brightness sum value, as the plane B's corrected angle.

Note that FIGS. 16 and 17 show an example where the second sectional image I3 is not combined with a Doppler image. This is not a limitation. The processing circuitry 17 with the display controlling function 176 may synthetically combine the B-mode image with a Doppler image in the second sectional image I3.

The description of the first embodiment above has assumed the instances where a Doppler image is generated and used basically as it is in the Doppler image combination to the B-mode image. However, this is not a limitation. The Doppler image in the synthetic combination to the B-mode image may undergo processing such as removal of color representation from a certain portion thereof.

More specifically, and for example, it will be supposed that a B-mode short-axis image of a blood vessel has been synthetically combined with a Doppler image of a reduced opacity. The processing circuitry 17 with the analyzing function 173 determines whether or not an object having a high brightness beyond a preset brightness value is detected in the short-axis image. The object having a higher brightness value than the preset brightness value here means, for example, the tip of the puncture needle that has reached the center of the blood vessel in the puncture operation. Upon detecting the needle tip in the short-axis image, the processing circuitry 17 with the display controlling function 176 removes the color representation of the combined Doppler image, from a predetermined range surrounding the detected needle tip.

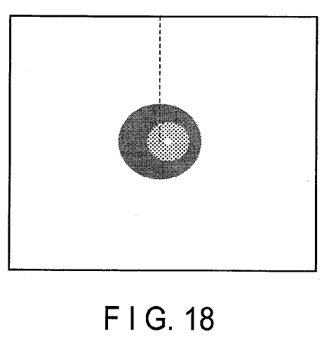

FIG. 18 is a schematic diagram showing an example of a display after the removal of the color representation of the Doppler image from an area surrounding the needle tip. As shown in FIG. 18, the color representation of the Doppler image has been removed from the area surrounding the high-brightness object, i.e., the needle tip, appearing in the vicinity of the center of the short-axis image.

### Second Embodiment

The first embodiment has been described based on the examples and instances of the ultrasound diagnostic apparatus 1 without regard to the compatibility with a needle navigation system. For the second embodiment, description will be given of an ultrasound diagnostic apparatus 1a compatible with needle navigation systems.

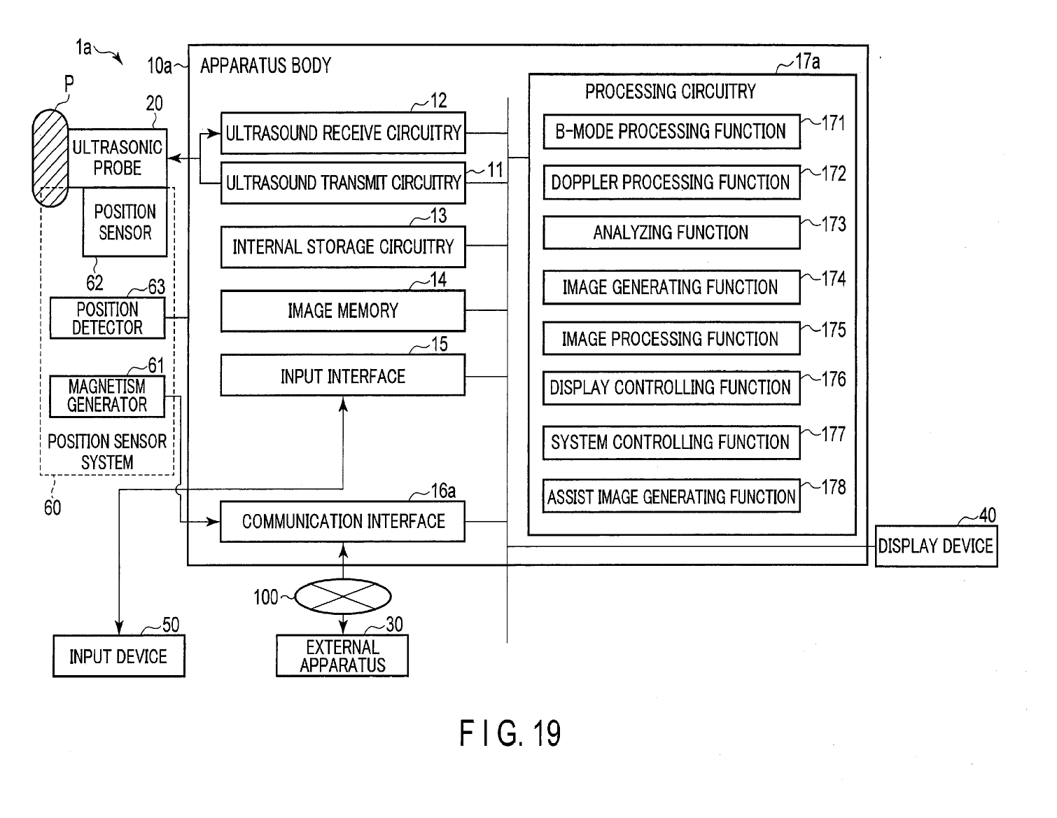

FIG. 19 is a block diagram showing an exemplary configuration of the ultrasound diagnostic apparatus 1a according to the second embodiment. As shown in FIG. 19, the ultrasound diagnostic apparatus 1a includes an apparatus body 10a, the ultrasound probe 20, and a position sensor system 60.

The position sensor system 60 is a system for acquiring information about the three-dimensional positions of the ultrasound probe 20 and the puncture needle. The position sensor system 60 includes, for example, a magnetism generator 61, one or more position sensors 62, and a position detector 63. The magnetism generator 61 includes, for example, a magnetism generating coil, etc. The magnetism generator 61 may be arranged at any position and is adapted to form a magnetic field spreading outward from itself.

Each position sensor 62 may be, for example, a magnetic sensor adapted to detect the strength and the gradient of the three-dimensional magnetic field formed by the magnetism generator 61. The position sensor 62 may be attached to one or each of the ultrasound probe 20 and the puncture needle. The position sensor 62 is adapted to output the detected strength and gradient of the magnetic field to the position detector 63.

The position detector 63 is adapted to calculate the positions of the ultrasound probe 20 and the puncture needle in a three-dimensional space defined by a predetermined point of origin, based on the strength and the gradient detected by the one or more position sensors 62. The predetermined point here is set at, for example, the position where the magnetism generator 61 is arranged. The position detector 63 is adapted to send the position information of the calculated positions to the apparatus body 10a.

A communication interface 16a in this embodiment is connected to the external apparatus 30 via the network 100, etc. as well, so that data communication is performed with the external apparatus 30. The communication interface 16a is also adapted to receive the position information for the ultrasound probe 20 and the position information for the puncture needle, sent from the position detector 63.

The ultrasound diagnostic apparatus 1a includes processing circuitry 17a adapted to execute the programs stored in the internal storage circuitry 13 to realize functions corresponding to the respective programs. The processing circuitry 17a further has, for example, an assist image generating function 178.

The assist image generating function 178 is a function for generating assist images based on the relative positional relationship between the ultrasound probe 20 and the puncture needle, using the position information acquired by the position sensor system 60. More specifically, the processing circuitry 17a with the assist image generating function 178 calculates the point for inserting the puncture needle based on the distance between the blood vessel center and the body surface, calculated by the analyzing function 173. The processing circuitry 17a then generates the assist images which may each indicate the blood vessel center, the insertion point of the puncture needle, and the current position of the puncture needle.

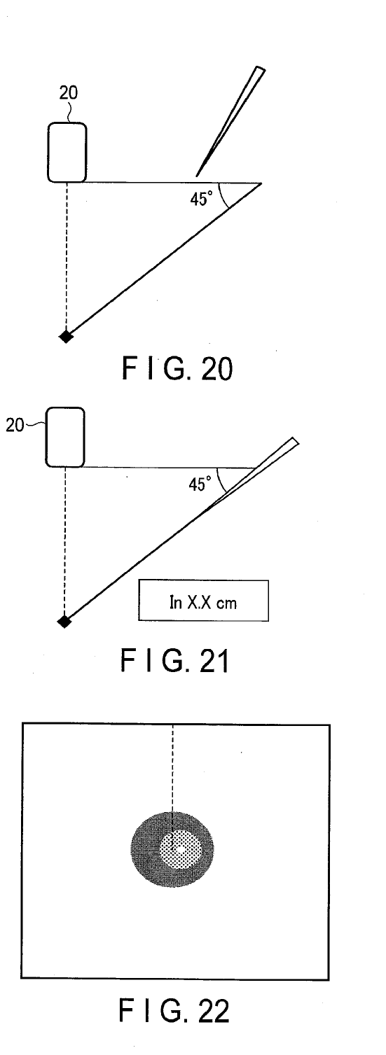

FIG. 20 is a schematic diagram showing an example of the assist images generated by the assist image generating function 178. As shown in FIG. 20, a guide graphics image is displayed to show the blood vessel center and the insertion point of the puncture needle, together with the position of the tip of the puncture needle before the puncture operation. This display allows the physician to confirm the position and the angle of the puncture needle in the current state, before proceeding with the insertion of the puncture needle.

FIG. 21 is a schematic diagram showing another example of the assist images generated by the assist image generating function 178. As shown in FIG. 21, a guide graphics image is displayed to show the blood vessel center and the insertion point of the puncture needle, together with the position of the tip of the puncture needle in the middle of the puncture operation. This display allows the physician to confirm the moving direction of the puncture needle and the inserted length of the puncture needle, during the ongoing puncture operation. It is possible for the image of FIG. 21 to additionally show the remaining distance to the blood vessel center, i.e., how far to reach the target.

The ultrasound diagnostic apparatus 1a compatible with needle navigation systems can remove color representation from a portion of the Doppler image synthetically added to the B-mode image, by performing the following processing, for example. The processing circuitry 17a with the analyzing function 173 determines whether or not the tip of the puncture needle has reached the blood vessel center based on the relative positional relationship between the ultrasound probe 20 and the puncture needle, using the position information sent from the position sensor system 60. Upon determining that the tip of the puncture needle has reached the blood vessel center, the processing circuitry 17a with the display controlling function 176 removes the color representation of the Doppler image added to the B-mode blood vessel image, from a predetermined range surrounding the needle tip.

FIG. 22 is a schematic diagram showing an example of a display after the removal of the color representation of the Doppler image from the predetermined range surrounding the needle tip. As shown in FIG. 22, the color representation of the Doppler image has been removed from the area surrounding the tip of the puncture needle having reached the vicinity of the center of the short-axis image.

According to the second embodiment as described, the ultrasound probe 20 carries out ultrasound scanning for the scan region in the body of a subject. The processing circuitry 17a of the ultrasound diagnostic apparatus 1a analyzes the result of the ultrasound scanning for the part corresponding to the central portion of the scan region, so that it calculates the distance between the blood vessel within the central portion and the body surface. The processing circuitry 17a then causes the display device 40 to display the calculated distance in real time. The processing circuitry 17a further generates assist images based on the calculated distance, and the relative positional relationship between the ultrasound probe 20 and the puncture needle, acquired in cooperation with the position sensor system 60. The ultrasound diagnostic apparatus 1a can therefore keep a physician from making an error in specifying the depth of a puncture, while allowing the physician to confirm various information for the state of the puncture needle, including the insertion point and the angle of the puncture needle.

According to at least one of the embodiments described above, the ultrasound diagnostic apparatus 1, as well as the ultrasound diagnostic apparatus 1a, can enable a physician to conduct puncture operations with enhanced usability and safety.

The term “processor” used in the descriptions of the embodiments refers to, for example, a central processing unit (CPU) or a graphics processing unit (GPU), or various types of circuitry which may be an application-specific integrated circuit (ASIC), a programmable logic device (such as a simple programmable logic device (SPLD), a complex programmable logic device (CPLD), or a field programmable gate array (EPGA)), and so on. The processor reads programs stored in the storage circuitry and executes them to realize the respective functions. The programs may be incorporated directly in circuits of the processor, instead of being stored in the storage circuitry. In this case, the processor reads the programs incorporated in its circuits and executes them to realize the functions. Each processor described in the embodiments is not limited to a single circuitry-type processor. A plurality of independent processors may be combined and integrated as one processor realizing multiple functions. Furthermore, multiple structural elements of the above embodiments may be integrated as one processor realizing multiple functions.

While certain embodiments have been described, they have been presented by way of example only, and are not intended to limit the scope of the inventions. Indeed, the novel embodiments described herein may be embodied in a variety of other forms. Furthermore, various omissions, substitutions, and changes in the form of the embodiments may be made without departing from the spirit of the inventions. The accompanying claims and their equivalents are intended to cover such forms or modifications as would fall within the scope and the spirit of the inventions.

1. An ultrasound diagnostic apparatus comprising:
an ultrasound probe adapted to be applied on a surface of a subject and configured to perform ultrasound scanning for a scan region in the subject; and
processing circuitry configured to
analyze, among a result of the ultrasound scanning, a part corresponding to a central portion of the scan region to calculate a distance between a blood vessel in the central portion and the surface, and
cause a display to display the distance, a value based on the distance, or both the distance and the value.

2. The ultrasound diagnostic apparatus according to claim 1, wherein
the ultrasound scanning comprises a scan for acquiring Doppler image data for a region of interest in the scan region, and
the processing circuitry is configured to calculate the distance by analyzing, among a result of the scan, a part corresponding to the central portion.

3. The ultrasound diagnostic apparatus according to claim 1, wherein
the ultrasound scanning comprises a first scan for acquiring B-mode image data for the scan region, and a second scan for acquiring Doppler image data for a region of interest in the scan region, and
the processing circuitry is configured to calculate the distance by analyzing, among a result of the first scan or the second scan, a part corresponding to the central portion.

4. The ultrasound diagnostic apparatus according to claim 1, wherein
the ultrasound scanning comprises a scan for acquiring B-mode image data for the scan region, and
the processing circuitry is configured to calculate the distance by analyzing, among a result of the scan, a part corresponding to the central portion.

5. The ultrasound diagnostic apparatus according to claim 1, wherein
the value is the distance multiplied by √2.

6. The ultrasound diagnostic apparatus according to claim 1, wherein
the processing circuitry is configured to cause the display to display neither the distance nor the value, if the central portion of the scan region does not comprise the blood vessel.

7. The ultrasound diagnostic apparatus according to claim 1, wherein
the processing circuitry is configured to cause the display to display a measurement line extending from the blood vessel in the central portion to the surface.

8. The ultrasound diagnostic apparatus according to claim 7, wherein
the processing circuitry is configured to cause the display to display none of the distance, the value, or the measurement line, if the central portion of the scan region does not comprise the blood vessel.

9. A method for assisting a puncture, comprising:
analyzing, among a result of ultrasound scanning by an ultrasound probe for a subject, a part corresponding to a central portion of a scan region of the ultrasound scanning to calculate a distance between a blood vessel in the central portion and a surface of the subject, and
causing a display to display the distance, a value based on the distance, or both the distance and the value.

10. The method according to claim 9, wherein
the ultrasound scanning comprises a scan for acquiring Doppler image data for a region of interest in the scan region, and
the distance between the blood vessel and the surface is calculated by analyzing, among a result of the scan, a part corresponding to the central portion.

11. The method according to claim 9, wherein
the ultrasound scanning comprises a first scan for acquiring B-mode image data for the scan region, and a second scan for acquiring Doppler image data for a region of interest in the scan region, and
the distance between the blood vessel and the surface is calculated by analyzing, among a result of the first scan or the second scan, a part corresponding to the central portion.

12. The method according to claim 9, wherein
the ultrasound scanning comprises a scan for acquiring B-mode image data for the scan region, and
the distance between the blood vessel and the surface is calculated by analyzing, among a result of the scan, a part corresponding to the central portion.

13. The method according to claim 9, wherein
the value is the distance multiplied by √2.

14. The method according to claim 9, further comprising
causing the display to display neither the distance nor the value, if the central portion of the scan region does not comprise the blood vessel.

15. The method according to claim 9, further comprising
causing the display to display a measurement line extending from the blood vessel in the central portion to the surface.

16. The method according to claim 15, further comprising
causing the display to display none of the distance, the value, or the measurement line, if the central portion of the scan region does not comprise the blood vessel.
